(19) United States
(12) Patent Application Publication
(10) Pub. No.: US 2010/0234477 A1
(43) Pub. Date: Sep. 16, 2010

(54) Catalyst and Process for Syngas Conversion

(75) Inventors: Xinhe Bao, Dalian (CN); Wei Chen, Dalian (CN); Xiulian Pan, Dalian (CN); Zhongli Fan, Dalian (CN); Yunjie Ding, Dalian (CN); Hongyuan Luo, Dalian (CN)

Correspondence Address: NIXON & VANDERHYE, PC, 901 NORTH GLEBE ROAD, 11TH FLOOR, ARLINGTON, VA, 22203, US

(73) Assignee: Dalian Institute of Chemical Physics, Chinese Academy of Sciences, Dalian China BP P.L.C., London (GB)

(21) Appl. No.: 12/223,917

(22) PCT Filed: Feb. 16, 2006
(86) PCT No.: PCT/CN2006/000228

§ 371 Date: Aug. 25, 2008

(51) Int. Cl. B01J 21/18; C07C 1/04; C07C 27/06

(52) U.S. Cl. 518/716; 502/185; 502/184; 977/742; 977/902

(57) ABSTRACT

A catalyst and process is described for the conversion of hydrogen and one or more oxides of carbon in which the catalyst comprises an elemental carbon-containing support. Also described is a process for reducing agglomeration in carbon nanotubes, in which carbon nanotubes are suspended in a liquid and simultaneously treated by ultrasound and agitation. The method can be used to prepare carbon nanotube-supported catalysts that show high activity towards the conversion of feedstocks comprising hydrogen and one or more oxides of carbon.

This invention relates to the field of catalysis, in particular to catalysts suitable for the conversion of hydrogen and one or more oxides of carbon.

Increasing attention is being paid to ethanol as a gasoline oxygenate additive. Ethanol is typically produced either by fermentation processes, for example from sugar beet or cane, or synthetically by ethylene hydration. However, in certain parts of the world, current production of ethanol by existing processes may not be able to meet with expected demand if it is to be used for blending with gasoline. There is therefore a need for an alternative process for producing ethanol or other oxygenated compounds in volumes sufficient to meet with expected demand.

Syngas (a mixture of hydrogen and carbon monoxide) can be used as a feedstock for the production of liquid hydrocarbon fuels or oxygenated organic compounds such as methanol or ethanol. Syngas can be produced by processes such as steam reforming, autothermal reforming or partial oxidation from a variety of substrates, such as natural gas, coal or biomass. It is therefore potentially available in extremely large quantities, and hence could be an attractive option to produce ethanol or other oxygenated compounds in high volumes.

EP-A-0 010 295 describes a process for preparing ethanol from synthesis gas, in which the reaction is carried out over a rhodium catalyst comprising, as co-catalyst, one or more of the elements zirconium, hafnium, lanthanum, platinum, chromium and mercury supported on a carrier such as a silicate or alumina.

EP-A-0 079 132 relates to a process for preparing oxygenated hydrocarbons by catalytic reaction of synthesis gas over a supported catalyst comprising, as active components, rhodium, silver, zirconium and molybdenum and also, if desired, iron, manganese, rhenium, tungsten, ruthenium, chromium, thorium and potassium. The preferred support material is silicon dioxide.

JP 62/148437 and JP 62/148438 disclose the simultaneous production of acetic acid, acetaldehyde and ethanol from a synthesis gas reacted in the presence of a rhodium catalyst pre-treated with sulphur-containing compounds. JP 61/178,933 discloses producing oxygenates from a synthesis gas wherein the reaction is carried out in the presence of a rhodium catalyst provided with an accelerator metal such as scandium, iridium or an alkali earth metal. JP01/294643 discloses the production of oxygenated compounds such as acetic acid in which a synthesis gas is reacted in the presence of a rhodium catalyst on a silica substrate.

U.S. Pat. No. 6,346,555 and U.S. Pat. No. 6,500,781 disclose a catalyst and a process for preparing C₂-oxygenates by reaction of CO and H₂ over a rhodium-containing supported catalyst, in which the catalyst consists essentially of rhodium, zirconium, iridium, at least one metal selected from amongst copper, cobalt, nickel, manganese, iron, ruthenium and molybdenum, and at least one alkali metal or alkaline earth metal selected from amongst lithium, sodium, potassium, rubidium, magnesium and calcium, on an inert support.

In the above-cited art, there is no disclosure of the performance of catalysts comprising carbon-containing supports, attention being mainly focussed on inorganic oxides as supports, such as silica or aluminosilicate zeolites. In U.S. Pat. No. 4,014,913 and U.S. Pat. No. 4,096,164, which describe processes for the conversion of syngas to C₂-oxygenates in the presence of catalysts comprising rhodium and manganese, or rhodium and molybdenum and/or tungsten respectively, it is taught that silica is a preferred support, while activated carbon is a least preferred support.

The use of carbon nanotubes as catalyst supports is known. For example, Zhang et al in Applied Catalysts A: General 187 (1999), 213-224 describe a Rh-phosphine catalyst supported on carbon nanotubes for propene hydroformylation, the catalyst being prepared by an incipient wetness technique from HRh(CO)(PPh₃)₃ complex in benzene. Giordano et al, in Eur. J. Inorg. Chem. 4 (2003), 610-617, describe the pre-treatment of multi-walled carbon nanotubes (MWNT) with nitric acid, and their subsequent use to support [Rh₂Cl₂(CO)₄]. The catalyst is also modified with sodium to produce surface bearing sodium carboxylate groups. This is stated to improve dispersion of the rhodium catalyst supported thereon. Hiura et al. in Adv. Mater. 7 (1995), 275-276, report that the effect of the nitric acid treatment is to form oxygen-containing groups on the surface of the carbon nanotubes, such as hydroxyl or carboxylate groups. It is further stated by Tsang et al. in Nature 372 (1994) 159-162 that nitric acid treatment removes the ends or tips of the nanotubes, which allows metal species to be loaded inside.

However, a problem when preparing carbon nanotube-supported catalysts is that the carbon nanotubes can agglomerate when suspended in a liquid or impregnating solution, resulting in poor separation of the carbon nanotubes, which reduces the surface area of the carbon nanotubes that are exposed to the impregnating solution.

There remains a need for catalysts with improved activity towards the conversion of feedstocks comprising hydrogen and one or more oxides of carbon. There also remains a need for an improved process and catalyst for the production of oxygenated compounds with two or more carbon atoms from hydrogen and one or more oxides of carbon. There also remains a need for an improved process for supporting catalyst components onto carbon nanotubes that reduces or eliminates the problem of nanotube agglomeration.

According to a first aspect of the present invention, there is provided a process for reducing agglomeration of carbon nanotubes comprising suspending carbon nanotubes in a liquid, characterised by the suspension being treated by a combination of ultrasound and agitation.

Treating carbon nanotubes suspended in a liquid by a combination of both ultrasound and agitation, for example during impregnation of the carbon nanotubes with one or more catalyst components, acts to prevent or reverse agglomeration of the carbon nanotubes, and increases the extent of separation of the nanotubes. This allows a higher surface area of the carbon nanotubes to be exposed to an impregnating solution.

The carbon nanotubes can be either single-walled or multi-walled nanotubes. Typically, carbon nanotubes have an inner diameter in the range of from 0.2 to 120 nm. For single-walled nanotubes, the inner diameters will typically be in the range of from 0.2 to 2 nm and outer diameters typically from 0.5 to 3 nm. For multi-walled nanotubes, the inner diameters will typically be in the range of from 0.5 to 120 nm and the outer diameters typically in the range of from 2 to 200 nm. When freshly prepared, the carbon nanotubes have a length typically in the range of from 0.5 to 200 μm.

The carbon nanotubes are preferably treated by oxidation so as to impart surface oxygen groups, such as hydroxyl, carbonyl and carboxyl groups onto the carbon nanotubes. Such treatment can also remove the tips of the carbon nanotubes, which allows the internal surface of the nanotubes to be functionalised with surface oxygen groups. The oxidising treatment can be carried out either simultaneously with the ultrasound and agitation treatment, or beforehand. The oxidation is typically achieved by suspending the nanotubes in a suitable oxidising agent, such as a solution of nitric acid, hydrogen peroxide solution, or a mixture of nitric and sulphuric acids. Preferably the treatment is carried out in aqueous nitric acid, with a nitric acid concentration preferably in the range of from 10 to 90 wt %, more preferably in the range of from 30 to 80 wt %, even more preferably in the range of from 60 to 80 wt %.

Ultrasound treatment may be continuous or pulsed. Typically, one or more frequencies in the range of from 15 to 100 kHz are used. This can be achieved using a water-filled ultrasound bath for example, or by inserting an ultrasound emitter, such as an ultrasound horn, into the suspension.

The ultrasound treatment additionally assists in removing any gas, such as air, that may be entrapped within the carbon nanotubes. Entrapped gas can otherwise act as a barrier to the suspending liquid, for example during oxidation treatment or catalyst impregnation. Thus, by removing the entrapped gas, the suspending liquid is more easily able to contact the internal surface of the carbon nanotubes, which facilitates the formation of surface oxygen groups on the internal surfaces during oxidation treatment, and can help improve impregnation and dispersion of one or more catalyst components within the carbon nanotubes.

The suspension of carbon nanotubes is additionally agitated. Preferably, this is achieved by stirring, for example using a magnetic stirrer or a manually or electrically operated paddle, blade or propeller stirrer. The combined effect of agitation and ultrasound treatment reduces the extent of carbon nanotube agglomeration to a greater extent than using just of one of the techniques alone. Thus, there is a synergistic effect in the combination of agitation and ultrasound treatment which unexpectedly enhances the extent of carbon nanotube separation, and enables the formation of a stable and homogeneous suspension with little settling of carbon nanotube particles. The agitation and ultrasound may be performed either simultaneously or sequentially.

The ultrasound treatment in combination with the agitation is carried out for a length of time sufficient to reduce carbon nanotube agglomeration to a sufficient extent, but without prolonging the treatment longer than is necessary. Typically, the length of time of the ultrasound treatment will be in the range of from 0.1 to 24 hours, preferably in the range of from 0.1 to 5 hours, and most preferably in the range of from 0.1 to 2 hours. Treating the impregnating solution for too long can result in an increase in agglomeration. Agitation is preferably performed for a longer period of time than ultrasound treatment, such as in the range of from 0.1 to 50 hours, preferably in the range of from 0.5 to 10 hours.

The weight ratio of the carbon nanotubes to the suspending liquid is suitably in the range of from 1:10 to 1:2000, preferably in the range of from 1:10 to 1:500. Most preferably, the range is from 1:50 to 1:300.

In one embodiment of the present invention, the oxidising treatment is carried out before the agitation and ultrasound treatment by suspending the carbon nanotubes in nitric acid and heating to boiling point while under reflux. This increases the extent of tip removal of the carbon nanotubes, and also removes residual amorphous carbonaceous material that may result from the initial synthesis of the carbon nanotubes. Additionally, the oxidising treatment can also shorten the carbon nanotubes, which further improves accessibility to the internal surfaces. The oxidising treatment is suitably carried out over a period of time in the range of from 0.1 to 100 hours, more preferably in the range of from 4 to 50 hours, even more preferably in the range of from 10 to 30 hours. Such treatment can reduce the length of the carbon nanotubes to a value typically in the range of from 300 to 800 nm.

In a preferred embodiment of the present invention, the combined ultrasound and agitation treatment is carried out on a suspension of carbon nanotubes in a liquid comprising one or more catalyst components. The presence of surface oxygen groups on the carbon nanotubes is advantageous when impregnating catalyst components onto a carbon nanotube as they can act as binding sites for catalyst components such as metals, and enables higher dispersion and increased loadings of the catalyst components to be achieved. Any oxidising treatment of the carbon nanotubes, either prior to or simultaneously with catalyst impregnation, can remove the tips and shorten the carbon nanotubes, which enables the impregnating solution to access both the internal and external surfaces of the carbon nanotubes, which further increases the quantity and dispersion of one or more catalyst components within the carbon nanotubes.

Carbon nanotube-supported catalyst is recovered from the suspension by methods such as filtration, decantation or evaporation to dryness. In a preferred embodiment, the supported catalyst is recovered from the suspension by evaporation of the liquid to dryness. The drying stage is preferably conducted so as to ensure an even distribution of the one or more catalyst components over the external and internal surfaces of the carbon nanotubes, and is preferably achieved by first evaporating the suspension to dryness at a temperature less than the boiling point of the liquid, followed by slowly ramping the temperature, either continuously or step-wise, to a temperature above the boiling point of the liquid. The evaporation is preferably carried out over a period of several hours, such as in the range from 10 to 72 hours. This ensures that the one or more catalyst components are deposited evenly throughout the internal and external surfaces of the carbon nanotubes, and reduces precipitation of large particles comprising the one or more catalyst components. In one embodiment of the invention, in which carbon nanotubes are suspended in an aqueous solution of one or more catalyst components, the suspension is allowed to evaporate to dryness at ambient temperature, before the temperature is slowly increased to a temperature above 100° C.

The one or more catalyst components are preferably metal-containing components which are able to bind to surface oxygen species described hitherto. Although it is possible to impregnate the carbon nanotubes using a dispersion or colloid of metal or metal-containing particles, it is preferred to use a solution of one or more metal compounds which are soluble in the suspending liquid, which improves the uniformity of impregnation throughout the carbon nanotubes.

Where there is more than one catalyst component, they may be impregnated onto the carbon nanotubes either simultaneously or sequentially. Preferably, the components are impregnated simultaneously using a solution comprising all the components in the desired concentrations, which reduces the number of impregnation steps required.

The liquid is preferably a hydrophilic liquid, which improves the dispersion and loading of one or more catalyst components that may be present in the liquid with hydrophilic surface oxygen groups that may be present on the carbon nanotubes. More preferably, the liquid is selected from water, an alcohol, a carboxylic acid, ethylene glycol or a mixture of two or more thereof.

The process of the present invention can be used for impregnating carbon nanotubes with metals such as alkali metals, alkaline earth metals or transition metals to produce, for example, a carbon nanotube-supported catalyst. Impregnation of the one or more catalyst components may be carried out either with a combined ultrasound and agitation treatment, or separately from the ultrasound and agitation treatment. Preferably, the impregnation is carried out in combination with the combined ultrasound and agitation treatment, as the reduced agglomeration of the carbon nanotubes ensures increased accessibility of the carbon nanotubes to the impregnating liquid. More preferably, impregnation of the one or more catalyst components is carried out either simultaneously with or after oxidation treatment of the carbon nanotubes, as removal of the tips of the carbon nanotubes and increasing the number of surface oxygen groups both on the interior and exterior surfaces of the carbon nanotubes improves the quantity and dispersion of the one or more impregnated catalyst components.

The one or more catalyst components that are impregnated onto the carbon nanotube support in accordance with the present invention can optionally be post-treated after removal of the impregnating liquid. For example, some supported metal catalysts are reduced before use to form supported metal particles, such as by exposure at elevated temperature to an inert atmosphere such as nitrogen or helium, or to a reducing atmosphere such as hydrogen. Supported metallic particles can sinter during reduction and when used as a catalyst in a reaction, such that the metal particles aggregate together on the support surface to form larger metal particles. This reduces the overall surface area of metal exposed to the reactants, and reduces catalyst activity. By supporting one or more catalyst metals in accordance with the method of the present invention, a higher quantity of catalyst metals can be impregnated inside the carbon nanotubes. When subsequently reduced to form catalyst metal particles, the size of the particles within the carbon nanotubes is restricted to the dimensions of the inner diameter of the carbon nanotube, which reduces sintering. Thus, by improving the quantity and dispersion of the impregnated catalyst metals during the impregnation stage, more catalyst metal particles will be formed within the carbon nanotube support, which improves catalyst activity and prolongs catalyst lifetime.

In one embodiment of the first aspect of the present invention, the carbon nanotube-supported catalyst can be used in reactions for the conversion of hydrogen and one or more oxides of carbon (for example syngas) into one or more organic compounds comprising at least one carbon atom in combination with hydrogen, such as hydrocarbons or oxygenated organic compounds.

An example of such a process is the production of liquid hydrocarbon fuels by Fischer-Tropsch synthesis. Such a process is suitably catalysed by catalysts comprising Fe, Co and/or Ni, preferably in the form of metallic particles.

Another example of a reaction for the conversion of hydrogen and one or more oxides of carbon is the production of oxygenated compounds comprising two or more carbon atoms from hydrogen and carbon monoxide, optionally also in the presence of carbon dioxide. Such catalysts preferably comprise rhodium, which is known to be active for such reactions. Preferably, the catalyst additionally comprises one or more elements selected from the group comprising alkali metals, Ti, V, Mn, Fe, Zr, Ru, Pd, Os, Ir and Pt. Even more preferably, the catalyst additionally comprises Mn, one of Li, Na or K, and at least one element selected from Ti, V, Fe, Zr, Ru, Pd, Os, Ir and Pt. Yet more preferably, the catalyst additionally comprises Mn, one of Li, Na or K, at least one element selected from Ti, V, Fe and Zr, preferably Ti, V and Fe, and at least one element selected from Ru, Pd, Os, Ir and Pt. In a particularly preferred embodiment of the invention, the catalyst comprises Rh, Mn, Li, Fe and Ir.

According to a second aspect of the present invention, there is provided a process for the conversion of hydrogen and one or more oxides of carbon into one or more organic compounds comprising at least one carbon atom in combination with hydrogen, which process comprises contacting hydrogen and carbon monoxide with a catalyst in a reaction zone, characterised in that the catalyst comprises an elemental carbon-containing support.

Elemental carbon-containing supports include activated carbon, carbon molecular sieves or carbon nanotubes. Preferably the catalyst comprises activated carbon or carbon nanotubes as the support. Without being bound by any theory, it is believed that the ability of elemental carbon to absorb hydrogen causes an increase in the concentration of hydrogen in the vicinity of one or more supported catalyst components, resulting in improved reactant conversions and product yields. Carbon nanotube-supported catalysts are particularly suited to such reactions, as carbon nanotubes typically have strong hydrogen-absorbing characteristics. Preferably, the carbon nanotubes and/or the carbon nanotube-supported catalyst is prepared using a process as hitherto described according to the first aspect of the present invention.

A mixture comprising hydrogen and carbon monoxide, optionally in the presence of carbon dioxide, is converted to one or more organic compounds comprising at least one carbon atom in combination with hydrogen. Examples of compounds comprising at least one carbon atom in combination with hydrogen according to the present invention include liquid hydrocarbons, such as those suitable for use as gasoline or gasoline additives, or those suitable for use as diesel or diesel additives. Other examples include oxygenated organic compounds, such as methanol, ethanol, ethyl acetate, acetic acid, acetaldehyde, or oxygenated compounds comprising three or more carbon atoms, such as C₃ or C₄ alcohols.

Preferably, carbon monoxide is one of the reactants. The molar ratio of hydrogen to carbon monoxide (H₂:CO) fed to the reaction zone is preferably in the range of from 0.1:1 to 20:1, more preferably in the range of from 1:1 to 5:1, and even more preferably in the range of from 1.5:1 to 2.5:1. The carbon monoxide and hydrogen can be fed separately to the reaction zone, or may be fed as a mixture. In a preferred embodiment, the source of carbon monoxide and hydrogen is syngas.

Preferably, the temperature of reaction zone is in the range of from 100 to 450° C., more preferably in the range of from 250 to 350° C. The pressure of the reaction zone is preferably in the range of from 1 to 200 bara (0.1 to 20 MPa), more preferably in the range of from 25 to 120 bara (2.5 to 12 MPa).

In a preferred embodiment, hydrogen and carbon monoxide, optionally also in the presence of carbon dioxide, are fed to a reaction zone comprising the catalyst at elevated temperature and pressure to form a product stream comprising one or more oxygenated compounds having two or more carbon atoms. The hydrogen and carbon monoxide may be fed separately to the reaction zone. Preferably, however, they are fed simultaneously, for example when using syngas as the feed to the reaction zone. Preferred products of the process of the present invention include one or more of ethanol, acetaldehyde, acetic acid and ethyl acetate. They are typically produced in combination with other oxygenated products, for example methanol or C₃ oxygenates such as i- or n-propanol, hydrocarbons such as methane, ethane and propane, and carbon dioxide.

The catalyst preferably comprises rhodium. Preferably, the rhodium catalyst additionally comprises one or more elements selected from the group comprising Ti, V, Mn, Fe, Zr, Ru, Pd, Os, Ir and Pt, and more preferably also an alkali metal. Even more preferably, the catalyst comprises Rh, Mn, one of Li, Na or K, and at least one element selected from Ti, V, Fe, Zr, Ru, Pd, Os, Jr and Pt. Yet more preferably, the rhodium catalyst additionally comprises Mn, one of Li, Na or K, at least one element selected from Ti, V, Fe and Zr, preferably Ti, V and Fe, and at least one element selected from Ru, Pd, Os, Jr and Pt. In a particularly preferred embodiment of the invention, the catalyst comprises Rh, Mn, Li, Fe and Ir.

The reaction may be carried out in the gas-phase, wherein the mixture of hydrogen and carbon monoxide is passed over the catalyst in the vapour phase, and the products are also in the vapour phase. Alternatively, the products may be liquid-phase. For the production of oxygenates with two or more carbon atoms, the process is preferably operated in the gas-phase, in which the gas hourly space velocity (GHSV) is preferably maintained in the region of from 100 to 30,000 h⁻¹ (litres gas converted to standard temperature and pressure per litre of catalyst per hour). More preferably, the GHSV is at least 500 h⁻¹, and even more preferably is at least 1000 h⁻¹.

Optionally, the process is integrated with a syngas generation process, such that syngas is generated in a syngas reactor, and then fed to the reaction zone where it is converted into an organic compound comprising at least one carbon atom in combination with hydrogen. The feed to the syngas reactor can be a source of hydrocarbons derived from fossil fuels, such as one or more of natural gas, natural gas liquids, LPG, naphtha, refinery-off gas, vacuum residuals, shale oils, asphalts, fuel oils, coal, lignin or hydrocarbon-containing process recycle streams. Alternatively, the syngas may be produced from biomass. Preferably, the syngas source is methane, for example as derived from natural gas or from biomass decomposition. The methane may be substantially pure, or may contain impurities, such as other light hydrocarbons, for example ethane, propane and/or butanes.

Hydrocarbons may be converted into syngas by processes such as steam reforming, autothermal reforming or partial oxidation. The syngas produced in the syngas reactor may additionally comprise carbon dioxide. If produced in the syngas reactor, the carbon dioxide may be fed together with the syngas to the reaction zone for the conversion of hydrogen and one or more oxides of carbon, or may alternatively be removed from the syngas.

The invention will now be illustrated by reference to the following non-limiting Examples and by reference to the Figures, in which;

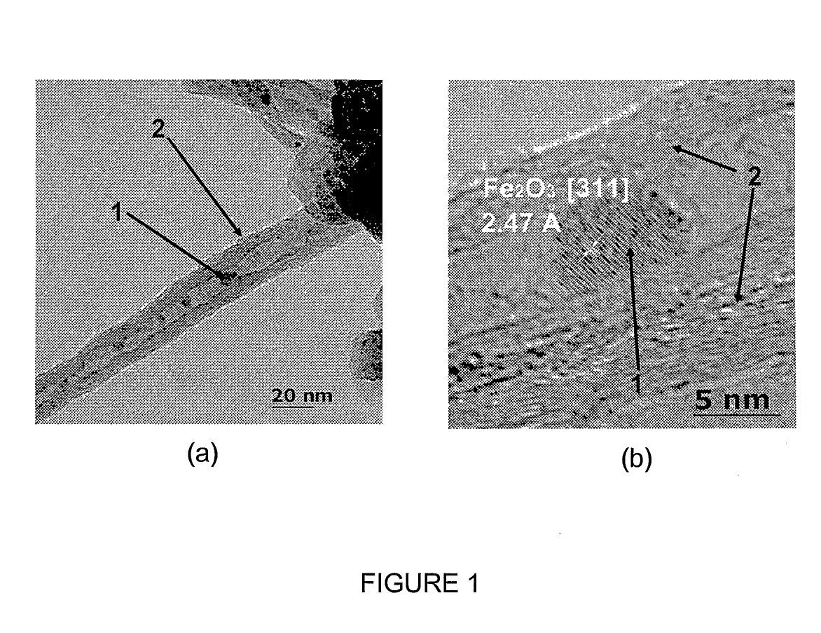

FIGS. 1a and b show TEM micrographs of iron oxide particles respectively within multiwalled carbon nanotubes, prepared by a process according to the first aspect of the present invention;

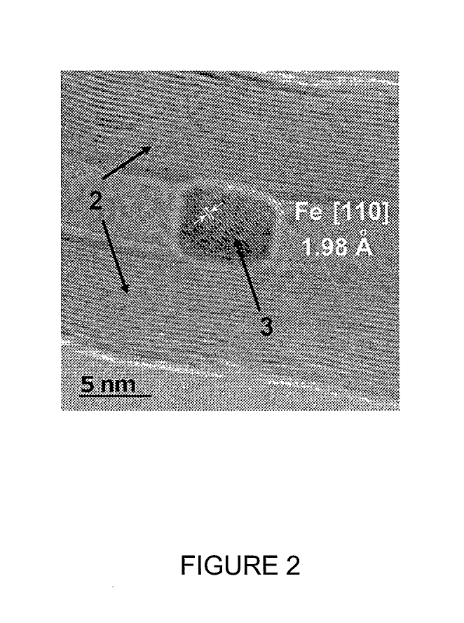

FIG. 2 shows a TEM micrograph of a reduced Fe(0) particle within a multiwalled carbon nanotube, prepared by reduction of carbon nanotube-supported iron oxide particles, and;

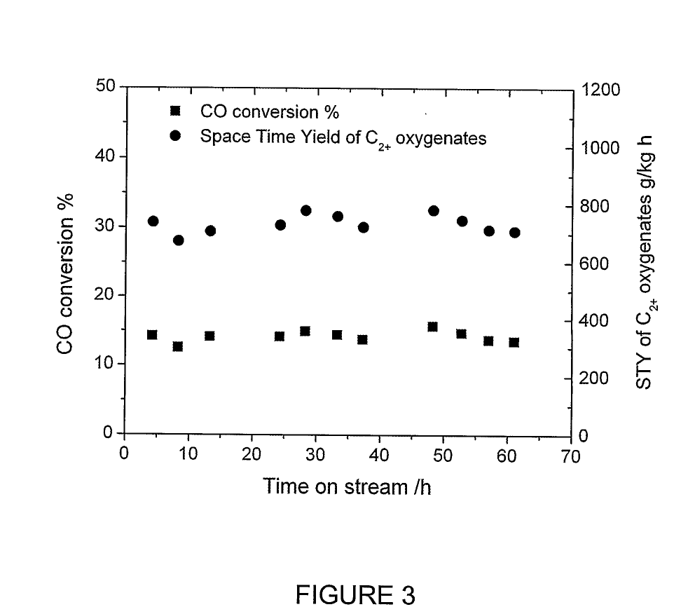

FIG. 3 is a graph showing catalyst activity data for a carbon nanotube-supported catalyst, prepared by a method according to the first aspect of the present invention, when used in the a process for the production of oxygenates with two or more carbon atoms from syngas, in accordance with the second aspect of the present invention.

### EXAMPLES

### Catalyst A

Carbon nanotubes (Chengdu Organic Chemicals Co. Ltd) were treated by being heated in 68 wt % nitric acid solution under reflux for 14 hours, before being filtered, washed with water and dried. Washing and drying were repeated until the wash-water had a pH value of 7. 0.5 g of the treated carbon nanotubes were then suspended in 50 mL an aqueous solution of RhCl₃, Mn(NO₃)₂, LiNO₃, Fe(NO₃)₃ and H₂IrCl₆, such that the weight ratio of Rh:Mn:Li:Fe:Ir:carbon nanotubes was 1.2:1.2:0.06:0.09:0.6:100 (metals as elements). The container comprising the suspension was placed in a water-filled ultrasound bath operating at a frequency of 23 kHz for 5 hours. The suspension was then continuously stirred under ambient conditions until the suspending solvent had evaporated.

The remaining solid was then dried at temperatures of 30, 40, 50 and 60° C., being held at each temperature for 3 hours. Finally, the sample was heated to 120° C. at a rate of 1° C./min and held at that temperature for 12 hours. This catalyst was prepared by a process in accordance with the first aspect of the present invention.

### Catalyst B

The same metals were supported on an activated carbon catalyst (Vulcan XC-72R from Cabot Corp.) in the same weight ratios as in Catalyst A. During impregnation, the suspension was stirred, but was not subjected to ultrasound treatment. The carbon was pre-treated with hydrochloric acid and subsequently with nitric acid before being suspended in the catalyst metal-containing solution.

### Catalyst C

Silica gel (Qingdao Haiyan Chemicals Group Corp.) was impregnated with the same catalyst metal-containing compounds as in Catalysts A and B, and in the same weight ratios, following the procedure described in CN 02160816. After drying, the catalyst was heated to a temperature of 120° C., and held at that temperature for 12 hours.

### Catalyst D

SBA-15 (Jilin University HighTech company Ltd, Changchun, China) was used as the support for the same metals and weight ratios as catalysts A, B and C, and was impregnated in the same way as catalyst C. SBA-15 is a silica comprising linearly arranged one-dimensional pores, with pore diameters in the range 6 to 7 nm.

### Catalyst E

Carbon nanotubes were pre-treated by nitric acid using an analogous process to that used in the preparation of catalyst A. The pre-treated carbon nanotubes were then suspended in a solution of FeCl₃ dissolved in a mixture of water and ethylene glycol. The suspension was treated first by ultrasound (30 minutes), followed by stirring with a magnetic stirrer for 4 hours. The pH of the suspension was increased to 8 using NaOH, and heated under reflux for 3 hours. The resulting suspension was filtered, washed with distilled water and dried overnight at 100° C. in air. The catalysts were then heated to 600° C. in a He stream, in order to produce reduced Fe(0) particles. This is an example of a catalyst prepared by a process according to the first aspect of the present invention.

FIGS. 1a and b show TEM micrographs of catalyst E before heating to 600° C. Iron oxide (Fe₂O₃) particles 1 are clearly shown to be located within the multiwalled carbon nanotubes 2. The catalyst shown has 80%±10% of the iron oxide particles located within the carbon nanotubes, the rest being on the outer surface. FIG. 2 shows a TEM micrograph of a typical iron oxide particle reduced to metallic Fe 3 within the multiwalled carbon nanotubes 2 by heating to 600° C. under a helium atmosphere.

These results show that metal particles impregnated into carbon nanotubes are able to remain within the carbon nanotubes after impregnation and reduction.

### Experiment 1

The activity of catalysts A to D, as described below, towards the synthesis of oxygenates having two or more carbon atoms from carbon monoxide and hydrogen was evaluated as follows. 0.4 g catalyst were packed into fixed-bed tube reactor. The catalyst was reduced in pure hydrogen at 350° C. for 2 hours, and the reactor was then cooled to the reaction temperature of 320° C. The hydrogen stream was turned off, and replaced by a syngas stream. Reaction pressure was 30 bara (3 MPa). The composition of the product stream from the reactor was analysed by gas chromatography. Results after 12 hours on stream are listed in table 1. Experiment 1, when using catalysts A and B, relates to a process according to the second aspect of the present invention because the catalysts comprise a carbon-containing support. Experiment 1, when using catalysts C and D, does not relate to a process according to the second aspect of the present invention as the catalysts do not have carbon-containing supports.

### Experiment 2

Catalyst A was used in a process similar to that used in experiment 1, except the pressure was held at 50 bara (5 MPa) over a period of 61 hours. FIG. 3 shows a plot of carbon monoxide conversion and yield of C₂₊ oxygenates (oxygenated compounds having two or more carbon atoms) with time on stream. No significant loss of activity was observed.

The results of Experiment 1 show that rhodium catalysts comprising a carbon-containing support (catalysts A and B) show superior CO conversions and product yields in the production of oxygenated organic compounds having two or more carbon atoms from syngas compared to silica supports.

Experiment 2 demonstrates that the activity of carbon nanotube-supported rhodium catalysts prepared in accordance the first aspect of the present invention remains stable even after several hours on stream.

TABLE 1

| | | | Selectivity to | Selectivity to | | |
|---|---|---|---|---|---|---|
| | CO | | C₂ | C₂₊ | STY C₂ | STY C₂₊ |
| | Conversion | Selectivity | Oxygenates | Oxygenates | Oxygenates | Oxygenates |
| Catalyst | (%)* | to CH₄ (%) | (%) | (%)** | (g/kg/h) | (g/kg/h)** |
| A | 13.88 | 17.82 | 36.68 | 47.36 | 519.70 | 649.40 |
| B | 6.99 | 17.48 | 39.02 | 45.43 | 271.20 | 310.97 |
| C | 4.46 | 34.71 | 43.56 | 53.60 | 194.86 | 235.57 |
| D | 2.05 | 27.31 | 39.22 | 47.92 | 80.08 | 96.64 |

*Reaction temperature 320° C., Reaction pressure 30 bara (3 MPa), Gas Hourly Space Velocity of reactants = 12500 h⁻¹.
**Oxygenated compounds comprising two or more carbon atoms.

1.-32. (canceled)

33. A catalyst composition comprising Rh on an elemental carbon-containing support, characterised in that the catalyst additionally comprises one or more elements selected from the group comprising Ti, V, Mn, Fe, Zr, Ru, Pd, Os, Ir and Pt.

34. A catalyst composition as claimed in claim 33, in which the catalyst additionally comprises an alkali metal.

35. A catalyst composition as claimed in claim 34 comprising Rh, Mn, one of Li, Na or K, and at least one element selected from Ti, V, Fe, Zr, Ru, Pd, Os, Ir and Pt.

36. A catalyst composition as claimed in claim 35 comprising Rh, Mn, one of Li, Na or K, at least one element selected from Ti, V, Fe, Zr, and at least one element selected from Ru, Pd, Os, Ir and Pt.

37. A catalyst composition as claimed in claim 36 comprising Rh, Mn, Li, Fe and Ir.

38. A catalyst composition as claimed in claim 33, in which one or more of the metals are in the form of reduced metal particles.

39. A catalyst composition as claimed in claim 33, in which the carbon-containing support is selected from activated carbon, carbon molecular sieve and carbon nanotubes.

40. A catalyst composition as claimed in claim 39, in which the carbon-containing support is selected from activated carbon and carbon nanotubes.

41. A catalyst composition as claimed in claim 40, in which the carbon-containing support is carbon nanotubes.

42. A process for producing a catalyst as claimed in claim 41, which process comprises suspending carbon nanotubes in a liquid and treating the suspension by a combination of ultrasound and agitation, characterised by the liquid being a solution of soluble catalyst components comprising Rh and one or more of Ti, V. Mn, Fe, Zr, Ru, Pd, Os, Ir and Pt.

43. A process as claimed in claim 42, in which agitation is achieved by stirring.

44. A process as claimed in claim 42, in which the carbon nanotubes are treated by oxidation so as to introduce surface oxygen groups, either simultaneously with the ultrasound or agitation treatment or beforehand.

45. A process as claimed in claim 44, in which the oxidation treatment is with aqueous nitric acid.

46. A process as claimed in claim 45, in which the pre-treatment is with aqueous nitric acid with a concentration of 60 to 80 wt %.

47. A process as claimed in claim 46, in which the oxidising pre-treatment is carried out at the boiling temperature of aqueous nitric acid under reflux.

48. A process as claimed in claim 44, in which the oxidation treatment of the carbon nanotubes is carried out before the agitation and ultrasound treatment.

49. A process as claimed in claim 42, in which the weight ratio of carbon nanotubes to the suspending liquid is in the range of from 1:10 to 1:500.

50. A process as claimed in claim 42, in which the suspending liquid is selected from water, an alcohol, a carboxylic acid, ethylene glycol or a mixture of two or more thereof.

51. A process as claimed in claim 42, in which a carbon nanotube-supported catalyst is recovered from the liquid.

52. A process as claimed in claim 51, in which one or more of the supported metals are reduced to form supported metal particles.

53. A process for the production of one or more organic compounds comprising at least one carbon atom in combination with hydrogen, which process comprises contacting hydrogen and carbon monoxide with a catalyst in a reaction zone, characterised in that the catalyst is a catalyst according to claim 33.

54. A process as claimed in claim 53, in which the hydrogen and carbon monoxide are converted into one or more oxygenated organic compounds having two or more carbon atoms.

55. A process as claimed in claim 54, in which the one or more oxygenated compounds having two or more carbon atoms include one or more of ethanol, acetaldehyde, acetic acid and ethyl acetate.

56. A process as claimed in claim 53, in which the reaction zone operates at a temperature of from 100 to 450° C., and a pressure in the range of from 1 to 200 bara (0.1 to 25 20 MPa).
